(19) United States
(12) Patent Application Publication
(10) Pub. No.: US 2026/0158290 A1
(43) Pub. Date: Jun. 11, 2026

(54) Red Light Therapy Bodysuit Device

(71) Applicant: Desiree Chandler, Gilbert, AZ (US)

(72) Inventor: Desiree Chandler, Gilbert, AZ (US)

(21) Appl. No.: 19/034,698

(22) Filed: Jan. 23, 2025

Related U.S. Application Data
(60) Provisional application No. 63/730,109, filed on Dec. 10, 2024.

(51) Int. Cl. A61N 5/06

(52) U.S. Cl. CPC A61N 5/0616; A61N 2005/0627; A61N 2005/0636; A61N 2005/0652; A61N 2005/0659; A61N 2005/0663; A61N 2005/0664

(57) ABSTRACT

A red light therapy bodysuit device is provided. The device is comprised of a red light therapy bodysuit device designed to provide comprehensive full-body therapy for pain relief, inflammation reduction, and overall wellness. The device comprises a flexible, breathable bodysuit made from antimicrobial materials such as spandex and nylon, featuring mesh panels for ventilation and mobility. The device includes modular sections for targeted therapy, each activated independently via buttons, allowing users to focus on specific body regions. Integrated LED diodes emit wavelengths of 630-670 nm and 810-850 nm for effective therapy, with adjustable intensity levels and a hypoallergenic silicone barrier for safety and comfort. A rechargeable lithium-polymer battery powers the suit, and user-friendly controls enable customization of therapy sessions with built-in safety features, including an automatic shut-off timer and overheating sensors.

### CROSS-REFERENCE TO RELATED APPLICATION

The present application claims priority to, and the benefit of, U.S. Provisional Application No. 63/730,109, which was filed on Dec. 10, 2024, and is incorporated herein by reference in its entirety.

### FIELD OF THE INVENTION

The present invention relates generally to the field of red light therapy. More specifically, the present invention relates to a bodysuit that has a plurality of interior LEDs for red light therapy effects while wearing the device. Accordingly, the present disclosure makes specific reference thereto. Nonetheless, it is to be appreciated that aspects of the present invention are also equally applicable to other like applications, devices, and methods of manufacture.

### BACKGROUND

Red light therapy has demonstrated significant potential in addressing various health conditions, including dental pain, hair loss, skin damage, and general inflammation. The therapy relies on specific wavelengths of red light that penetrate the skin and interact with cells to promote healing, reduce inflammation, and enhance cellular function. However, current methods of delivering red light therapy present numerous challenges. These methods typically involve stationary devices that require the user to remain in one fixed location while the therapy is applied to a specific part of the body. This lack of mobility is highly inconvenient for individuals with busy schedules, making it difficult to allocate the necessary time for regular therapy sessions. In many cases, the time commitment required for these stationary sessions deters potential users, leading to the progression of conditions such as chronic pain, inflammation, and delayed wound healing. Additionally, traditional devices are limited in coverage, treating only isolated areas rather than providing full-body benefits. As a result, users may not achieve comprehensive therapeutic outcomes. The need to travel to designated therapy locations further compounds the inconvenience and limits accessibility, particularly for those with mobility issues or limited transportation options. Without a more practical and accessible solution, inflammation, aches, and various types of pain may worsen over time, negatively impacting quality of life. Therefore, a solution that integrates red light therapy into a wearable, full-body format is necessary to improve accessibility, convenience, and therapeutic effectiveness.

Therefore, there exists a long-felt need in the art for the red light therapy bodysuit device that provides a convenient and portable solution for delivering red light therapy to multiple areas of the body simultaneously. There also exists a long-felt need in the art for the red light therapy bodysuit device that allows users to perform therapy while engaging in everyday activities. Moreover, there exists a long-felt need in the art for the red light therapy bodysuit device that offers a user-friendly, non-invasive, and time-efficient therapy method to alleviate pain, promote healing, and improve cellular function without side effects.

The subject matter disclosed and claimed herein, in one embodiment thereof, comprises a red light therapy bodysuit device. The device is comprised of a red light therapy bodysuit device designed to provide comprehensive full-body therapy for pain relief, inflammation reduction, and overall wellness. The device comprises a flexible, breathable bodysuit made from antimicrobial materials such as spandex and nylon, featuring mesh panels for ventilation and mobility. The device includes modular sections for targeted therapy, each activated independently via buttons, allowing users to focus on specific body regions. Integrated LED diodes emit wavelengths of 630-670 nm and 810-850 nm for effective therapy, with adjustable intensity levels and a hypoallergenic silicone barrier for safety and comfort. A rechargeable lithium-polymer battery powers the suit, and user-friendly controls enable customization of therapy sessions with built-in safety features, including an automatic shut-off timer and overheating sensors.

In this manner, the red light therapy bodysuit device of the present invention accomplishes all the foregoing objectives and provides red light therapy via red light emitters throughout the interior of a flexible, full-body suit. The suit enables the user to receive therapy while remaining mobile, thereby eliminating the inconvenience of stationary sessions. The inclusion of a rechargeable internal battery ensures portability, while the comfortable design allows the suit to be worn throughout daily activities. Additionally, the non-invasive red light therapy promotes cell recovery, reduces inflammation, and supports overall wellness, providing an efficient and side-effect-free therapeutic option for pain relief and healing.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a red light therapy bodysuit device. The red light therapy bodysuit device provides full-body red light therapy for pain relief, inflammation reduction, and general wellness by covering multiple regions, including the head, torso, arms, and legs. The device comprises a bodysuit made of flexible, breathable, antimicrobial materials such as spandex, nylon, and moisture-wicking synthetic fibers. These materials ensure a comfortable fit and allow full mobility while maintaining hygiene.

In one embodiment, the bodysuit features at least one mesh panel positioned in high-movement areas like the elbows, knees, shoulders, and hips. These panels improve ventilation, enhance flexibility, and facilitate movement. The bodysuit includes a front fastener, such as a zipper, hook-and-loop closures, or magnetic fasteners, for case of wear and removal.

The bodysuit comprises modular sections that enable independent activation of specific body regions. These sections may include a head section for cranial and cervical areas, a torso section for the chest, back, and abdomen, an arm section extending to the hands, and a leg section covering the hips, thighs, knees, calves, and feet. Each section can be activated using at least one button, allowing for customized therapy targeting specific areas.

The interior of the bodysuit contains at least one LED diode emitting wavelengths of 630-670 nm for red light and 810-850 nm for near-infrared light. The diodes are arranged for even light distribution, and their intensity can be adjusted to low, medium, or high via the button. A clear hypoallergenic silicone barrier between the diodes and the skin ensures safety, prevents irritation, and enhances light penetration.

Power is supplied by a lightweight lithium-polymer battery housed in a pocket on the lower back or hip for balanced weight distribution. The battery is rechargeable via a charging port, such as a USB-C connector. The button also controls power, light intensity, and timer functions for precision therapy. Safety features of the device include an automatic shut-off timer settable to 15, 30, or 60 minutes to prevent overexposure and built-in sensors that detect overheating, pausing the therapy session if necessary.

The device offers a practical solution for red light therapy with full-body coverage, customizable options, and safety features. The combination of modular design, advanced technology, and user-friendly controls enhances accessibility, convenience, and effectiveness for regular wellness routines.

The method of use involves donning the bodysuit by opening the front fastener and aligning the modular sections with corresponding body regions. The device is activated by pressing the button to power on and select specific sections for therapy. The LED intensity can be adjusted, and a timer set for 15, 30, or 60 minutes. The device then delivers red light therapy through the LEDs, emitting specified wavelengths for therapeutic benefit.

Accordingly, the red light therapy bodysuit device of the present invention is particularly advantageous as it provides red light therapy via red light emitters throughout the interior of a flexible, full-body suit. The suit enables the user to receive therapy while remaining mobile, thereby eliminating the inconvenience of stationary sessions. The inclusion of a rechargeable internal battery ensures portability, while the comfortable design allows the suit to be worn throughout daily activities. Additionally, the non-invasive red light therapy promotes cell recovery, reduces inflammation, and supports overall wellness, providing an efficient and side-effect-free therapeutic option for pain relief and healing.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of one potential embodiment of a red light therapy bodysuit device of the present invention in accordance with the disclosed architecture; and

FIG. 2 illustrates a flowchart of a method of using one potential embodiment of a red light therapy bodysuit device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for the red light therapy bodysuit device that provides a convenient and portable solution for delivering red light therapy to multiple areas of the body simultaneously. There also exists a long-felt need in the art for the red light therapy bodysuit device that allows users to perform therapy while engaging in everyday activities. Moreover, there exists a long-felt need in the art for the red light therapy bodysuit device that offers a user-friendly, non-invasive, and time-efficient therapy method to alleviate pain, promote healing, and improve cellular function without side effects.

The present invention, in one exemplary embodiment, is comprised of a red light therapy bodysuit device. The red light therapy bodysuit device provides full-body red light therapy to alleviate pain, reduce inflammation, and enhance overall wellness by covering multiple areas, including the head, torso, arms, and legs. The device comprises a bodysuit constructed from flexible, breathable, and antimicrobial materials such as spandex, nylon, and moisture-wicking synthetic fibers. These materials ensure a comfortable fit, allowing full mobility while maintaining hygiene.

In one embodiment, the bodysuit features at least one mesh panel located in high-movement areas, including the elbows, knees, shoulders, and hips. These panels improve ventilation, enhance flexibility, and facilitate movement. A front fastener, such as a zipper, hook-and-loop closures, or magnetic fasteners, allows for easy wearing and removal.

The bodysuit includes modular sections that can be activated independently to target specific body regions. These sections may include a head section for cranial and cervical areas, a torso section covering the chest, back, and abdomen, an arm section extending to the hands, and a leg section covering the hips, thighs, knees, calves, and feet. Each section can be activated via at least one button, allowing for customized therapy sessions.

The interior of the bodysuit incorporates at least one LED diode that emits wavelengths of 630-670 nm for red light and 810-850 nm for near-infrared light. The diodes are arranged for even light distribution, and their intensity can be adjusted to low, medium, or high using the button. A clear hypoallergenic silicone barrier between the diodes and the skin ensures safety, prevents irritation, and enhances light penetration.

A lightweight lithium-polymer battery supplies power to the bodysuit and is housed in a pocket on the lower back or hip to maintain balanced weight distribution. The battery can be recharged through a charging port, such as a USB-C connector. The button controls power, light intensity, and timer settings for precise therapy sessions. Safety features include an automatic shut-off timer adjustable to 15, 30, or 60 minutes to prevent overexposure and built-in sensors that detect overheating and pause the therapy session if necessary.

The device offers an efficient solution for red light therapy by providing full-body coverage, customizable options, and essential safety features. The combination of modular design, advanced technology, and user-friendly controls ensures convenience, accessibility, and effectiveness for regular wellness routines.

The method of use involves putting on the bodysuit by opening the front fastener and aligning the modular sections with the corresponding body regions. The device is activated by pressing the button to power it on and selecting specific sections for therapy. The LED intensity can be adjusted, and a timer can be set for 15, 30, or 60 minutes. The device then delivers red light therapy through the LEDs, emitting specified wavelengths for therapeutic benefits.

The red light therapy bodysuit device offers significant advantages by delivering therapy through emitters within a flexible, full-body suit. This design allows users to remain mobile during therapy sessions, eliminating the inconvenience of stationary treatments. The inclusion of a rechargeable internal battery ensures portability, while the comfortable design allows for wear during daily activities. The non-invasive red light therapy supports cell recovery, reduces inflammation, and promotes overall wellness, providing an effective and side-effect-free option for pain relief and healing.

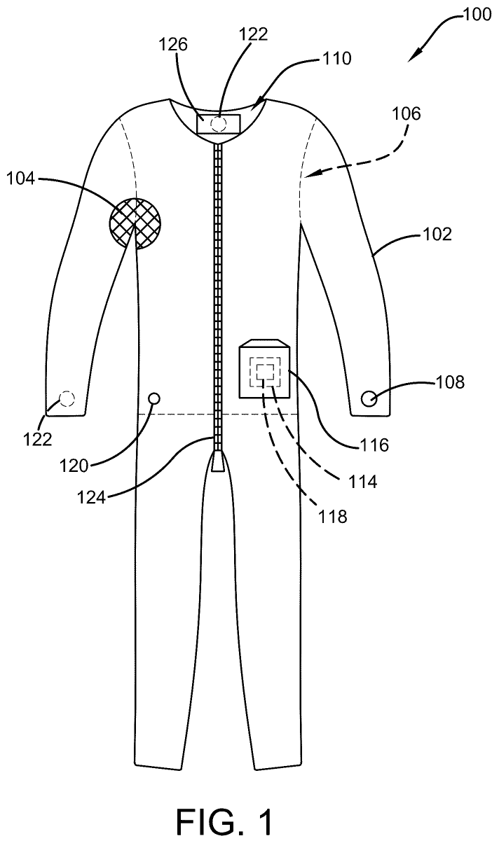

Referring initially to the drawings, FIG. 1 illustrates a front view of one potential embodiment of a red light therapy bodysuit device 100 of the present invention in accordance with the disclosed architecture. The red light therapy bodysuit device 100 provides a comprehensive, full-body solution for red light therapy, promoting pain relief, reducing inflammation, and supporting overall wellness by covering multiple body regions, including areas such as, but not limited to, the head, torso, arms, and legs. The device 100 is comprised of a bodysuit 102, which may be designed in various styles to cover different combinations of a user's body parts such as the arms, legs, hands, feet, torso, back, head, neck, and face. The bodysuit 102 is comprised of flexible, breathable, and antimicrobial materials, including, but not limited to, spandex, nylon, and moisture-wicking synthetic fibers. These materials are selected to provide a comfortable fit, allowing for full mobility during use while maintaining hygiene and reducing bacterial growth.

In one embodiment, the bodysuit 102 is comprised of at least one mesh panel 104 strategically placed in high-movement areas such as joints, including, but not limited to, the elbows, knees, shoulders, and hips. These mesh panels 104 enhance ventilation, promote air circulation, and increase flexibility, allowing the bodysuit to adapt to dynamic movements without restricting motion. To facilitate case of wear, the bodysuit 102 features a front fastener 124, which can include options such as a zipper, hook and loop closures, or magnetic fasteners. This front fastener 124 enables users to quickly and effortlessly donning and removal the bodysuit 110.

To provide versatility and targeted therapy, the bodysuit 102 is divided into modular sections 106, as seen in FIG. 1. These modular sections 106 allow users to activate specific regions independently, enabling therapy to be applied to selected body parts as needed. The modular sections 106 can include, but are not limited to, a head section that addresses the cranial and cervical regions, a torso section that covers the chest, back, and abdomen, an arm section that extends to the hands, and a leg section that covers the hips, thighs, knees, calves, and feet. Each section 106 can be activated independently through at least one button 108, allowing users to tailor their therapy sessions by focusing on specific areas of discomfort or inflammation. This modular design provides flexibility for customized treatments, accommodating a wide range of therapeutic needs.

The interior space 110 of the bodysuit 102 incorporates at least one LED diode 112 capable of emitting wavelengths between 630-670 nm for visible red light and/or 810-850 nm for near-infrared light. These specific wavelengths are effective for treating both superficial tissues and deeper muscle layers, enhancing the overall therapeutic benefit. The diodes 112 are preferably arranged in an even distribution throughout the bodysuit 102 to ensure comprehensive coverage and consistent light exposure. However, alternative arrangements of the diodes 112 may be used depending on design specifications or therapeutic requirements. The intensity of the diodes 112 can be adjusted to low, medium, or high using the button 108, allowing users to customize the intensity of the red light therapy based on their specific needs and comfort levels. Additionally, a clear hypoallergenic silicone barrier 126 is positioned between the diodes 112 and the user's skin to prevent irritation and ensure optimal light penetration, maintaining safety and comfort during use.

Power for the bodysuit 102 is supplied by a lightweight, high-capacity lithium-polymer battery 114, which is housed in a pocket 116 strategically positioned on the lower back or hip area, as seen in FIG. 1. This placement helps to balance the weight distribution of the bodysuit 102, preventing strain or discomfort during wear. The battery 114 can be recharged through a charging port 118, which may include options such as a USB-C charger. The button 108 also allows users to adjust power settings, light intensity, and timer functions, enhancing the user experience by providing precise control over therapy sessions.

To ensure user safety, the device 100 includes an automatic shut-off timer 120 that can be set for 15, 30, or 60 minutes to prevent overexposure to red light. Additionally, the bodysuit 102 incorporates built-in sensors 122 capable of detecting overheating via measuring temperature. If excessive heat is detected, the sensors 122 automatically pause the therapy session to prevent potential harm. These safety features enhance the reliability and security of the device, ensuring it can be used confidently for extended periods.

In summary, the red light therapy bodysuit device 100 offers a practical and versatile approach to red light therapy by providing full-body coverage, customizable therapy options, and safety features. The combination of a modular design, advanced red light technology, and user-friendly controls ensures an effective solution for pain relief, inflammation reduction, and overall wellness. This innovative device is designed for convenience, comfort, and ease of use, making red light therapy an accessible and valuable part of regular health and wellness routines.

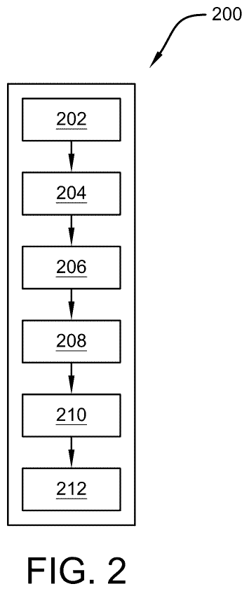

The red light therapy bodysuit device 100 is also comprised of a method of using 200 the device 100, as seen in FIG. 2. First, a device 100 is provided, wherein the device 100 comprises a bodysuit 102 comprised of modular sections 106, at least one LED diode 112, at least one button 108, a battery 114, and a clear hypoallergenic silicone barrier 126 positioned between the LED diode 112 and the skin [Step 202]. Then, a user dons the bodysuit 102 by opening the front fastener 124 and putting on the bodysuit 102 so that the modular sections 106 align with corresponding body regions such as the head, torso, arms, and legs [Step 204]. Following this, the user activates the bodysuit 102 by pressing at least one button 108 to power the device 100 and select the desired modular sections 106 for targeted therapy [Step 206]. The user can then adjust the LED diode 112 intensity to low, medium, or high, depending on individual therapeutic needs, by pressing the button 108 [Step 208]. Additionally, the user may configure the therapy session by setting a timer on the device 100 for 15, 30, or 60 minutes to prevent overexposure [Step 210]. The device 100 will then deliver red light therapy through the LED diodes 112, emitting wavelengths between 630-670 nm for visible red light and/or 810-850 nm for near-infrared light.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “red light therapy bodysuit device” and “device” are interchangeable and refer to the red light therapy bodysuit device 100 of the present invention.

Notwithstanding the foregoing, the red light therapy bodysuit device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the red light therapy bodysuit device 100 as shown in the FIGS. are for illustrative purposes only, and that many other sizes and shapes of the red light therapy bodysuit device 100 are well within the scope of the present disclosure. Although the dimensions of the red light therapy bodysuit device 100 are important design parameters for user convenience, the red light therapy bodysuit device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A red light therapy bodysuit device comprising:
a bodysuit configured to cover multiple body regions, the bodysuit comprised of a modular section configured to activate independently;
an LED diode positioned within the bodysuit and configured to emit a wavelengths
a button operatively connected to the modular section and the LED diode, the button configured to control an activation of the modular section and adjust the intensity of the LED diode; and
a battery operatively connected to the LED diode, the battery configured to supply power to the bodysuit.

2. The red light therapy bodysuit device of claim 1, wherein the wavelength emitted from the LED diode is between 630-670 nm.

3. The red light therapy bodysuit device of claim 1, wherein the wavelength emitted from the LED diode is between 810-850 nm.

4. The red light therapy bodysuit device of claim 1, wherein the bodysuit is comprised of a clear hypoallergenic barrier.

5. The red light therapy bodysuit device of claim 4, wherein the clear hypoallergenic barrier covers the LED diode.

6. The red light therapy bodysuit device of claim 1 further comprised of a sensor.

7. The red light therapy bodysuit device of claim 1, wherein an intensity of the LED diode is adjustable via the button.

8. The red light therapy bodysuit device of claim 7, wherein the LED diode is adjustable between a low, a medium, and a high setting.

9. The red light therapy bodysuit device of claim 1, wherein the bodysuit covers a head section, a torso section, an arm section, a back section, and a leg section of a user.

10. A red light therapy bodysuit device comprising:
a bodysuit configured to cover multiple body regions, the bodysuit comprised of a modular section configured to activate independently, a mesh panel, and a front fastener;
a plurality of LED diodes positioned within the bodysuit and configured to emit a wavelengths
a button operatively connected to the modular section and the plurality of LED diodes, the button configured to control an activation of the modular section and adjust the intensity of the plurality of LED diodes; and
a battery operatively connected to the plurality of LED diodes, the battery configured to supply power to the bodysuit.

11. The red light therapy bodysuit device of claim 10 further comprised of an automatic shut-off timer that deactivates the LED diode after a predetermined duration of use.

12. The red light therapy bodysuit device of claim 10, wherein the plurality of LED diodes are distributed evenly throughout the bodysuit.

13. The red light therapy bodysuit device of claim 10, wherein the bodysuit is comprised of a moisture-wicking material.

14. The red light therapy bodysuit device of claim 10, wherein the mesh panel is positioned at a joint area of the bodysuit.

15. The red light therapy bodysuit device of claim 10 further comprised of a sensor.

16. The red light therapy bodysuit device of claim 15, wherein the sensor is configured to detect overheating.

17. The red light therapy bodysuit device of claim 10, wherein the bodysuit is comprised of a clear hypoallergenic barrier.

18. The red light therapy bodysuit device of claim 10, wherein the battery is housed in a pocket.

19. A method of using a red light therapy bodysuit device, the method comprising the following steps:
providing a red light therapy bodysuit device comprised of a bodysuit comprising a modular section, an LED diode, a button, a battery, and a clear hypoallergenic silicone barrier positioned between the LED diode and a user's skin;
aligning the modular section of the bodysuit with a corresponding body region of a user;
activating a modular section by pressing the button;
adjusting the intensity of the LED diode using the button; and
delivering red light therapy through the LED diode to the body region of the user.

20. The method of using a red light therapy bodysuit device of claim 19 further comprised of a step of adjusting the intensity of the LED diode by selecting a low, a medium, or a high intensity level.
